(19) United States
(12) Patent Application Publication
(10) Pub. No.: US 2020/0176092 A1
(43) Pub. Date: Jun. 4, 2020

(54) Platform to Create System of Engagement for Health Needs

(71) Applicant: Presbyterian Healthcare Services Inc., Albuquerque, NM (US)

(72) Inventors: Mark Ries Robinson, Albuquerque, NM (US); Jessica Lin, Albuquerque, NM (US)

(21) Appl. No.: 16/699,018

(22) Filed: Nov. 28, 2019

Related U.S. Application Data
(60) Provisional application No. 62/772,676, filed on Nov. 29, 2018.

(51) Int. Cl. G16H 10/60; G16H 40/67; G06Q 10/10

(52) U.S. Cl. CPC G16H 10/60; G06Q 10/1057; G16H 40/67

(57) ABSTRACT

The invention relates to a method, system, and hardware that creates a differentiated patient/member experience by assessing the complexity of the patient's need and selecting the correct venue of communication. The invention further relates to a system facilitating development, deployment, and use of various applications using storefronts that reflect differing user, privacy, and information requirements of distinct user groups accessing a common data model for information related to health and health care.

### CROSSREFERENCE TO RELATED APPLICATIONS

This application claims the benefit of U.S. provisional 62/772,676, filed 29 Nov. 2018, which is incorporated herein by reference.

### TECHNICAL FIELD

The invention relates to a method, system, software, and hardware that provides a platform or operating system that integrates various systems of records to create an ecosystem of tools that improve the quality, cost and convenience of healthcare. The invention leverages a common data model to facilitate data interoperability and provide a shared data language for business and analytical applications to use. The Common Data Model simplifies data management and app (also referred to as application) development by unifying data into a known form and applying structural and semantic consistency across multiple apps and deployments. Healthcare represents a unique environment for application development as the purchaser, user, and the responsible entity may differ based on the application, but the applications access a common data model. The invention creates an application marketplace that recognizes these three distinct attributes of use and creates a mechanism for effective management, use and distribution of applications.

### SUMMARY OF INVENTION

Embodiments of the present invention provide methods, systems, software, and hardware that provide a differentiated level of digital integration for health system by creating a platform with a common data model that supports multiple application storefronts based on the end user and creates a system for privacy management, purchasing, and data monitoring responsibilities. The invention integrates information from systems of record to create a system of engagement tailored to the user of the application. Anticipated users include patients and members, employees of the health system, care staff and providers. The system controls access to data in a thoughtful and controlled manner, with awareness of health privacy concerns. The resulting platform creates a structured yet expandable system for the development of storefronts that share common attributes and creates an ecosystem upon which the future of digital health is created. Privacy management as used herein refers to the process and methods that protect and control sharing of a patient's health information.

The system can also contain a sophisticated communication element that proactively anticipates a user's questions and suggests the most effective mode of communication. The selection of a mode of communication can be based upon user characteristics, the complexity of the interaction, the type of information provided by the user, the type of information provided by the contact center, and combinations thereof. These factors can be assessed at the start of a conversation and updated based upon the flow of the interaction. These factors as well as any indications of frustration by the patient/member can be continuously evaluated so that an optimized interaction transpires. The system can determine the best venue of communication and via continuous dynamic assessment can recommend changes in communication venue based upon user interactions as well as the content of the interaction.

Embodiments of the invention provide a system for providing a plurality of health-related apps to a plurality of different types of users, comprising: (a) a common data model with access to at least two of the following: a directory of providers, patient medical records, a formulary, health insurance plan coverage records; (b) a plurality of storefronts that provide access to apps, wherein the apps are available in storefronts based on how the app implements privacy management of the patient's data and how the app implements secondary monitoring obligations relative to information from the app. In some embodiments, the plurality of storefronts comprises a first storefront wherein apps provide for privacy managed by a health system, and a second storefront wherein apps provide for privacy managed by the patient. In some embodiments, the plurality of storefronts comprises a first storefront wherein apps provide for secondary monitoring by a health system, and a second storefront wherein apps provide for data monitoring by a patient.

In some embodiments, the plurality of storefronts comprises at least two of the following: (1) a customer app storefront, accessible to individual health plan members, patients, or both, and containing apps that accept health-related data from the user and do not access PHI associated with any other individual; (2) a business operations storefront, accessible to individuals authorized by business operations of one or more health systems, and containing apps that access PHI of members of the health system; (3) a care management storefront, accessible to individuals authorized by a health care provider or health insurance plan, and containing apps that collect or allow input of PHI of the individual and communicate said PHI to a health care provider or health insurance plan; and (4) a provider storefront, accessible to one or more health care providers, and containing apps that access PHI of an individual from a health system, and communicate said PHI to one or more health care providers or health insurance plans.

In some embodiments, the common data model is configured to access a plurality of distinct data storage facilities, each controlled by a distinct health care system and containing PHI corresponding to members of the health care system. In some embodiments, the common data model uses software that translates stored health data and coordinates data retrieval actions between the common data model and the data storage model of the distinct storage facility to which the data storage or data retrieval action pertains.

In some embodiments, an individual can access an app from the care management storefront upon receiving a prescription for the app from a health care provider.

### BRIEF DESCRIPTION OF THE DRAWINGS

FIG. 1 is a schematic illustration of connectivity between applications and databases.

FIG. 2 is a schematic illustration of connectivity between applications and databases using a shared data model.

FIG. 3 is a table illustrating distinct attribute differences between multiple storefronts.

FIG. 4 is a schematic illustration of an example system showing the current interconnections of an example system without benefit of the present invention and how the associated parts communicate.

FIG. 5 is a schematic illustration of an example system showing similar functionality as in FIG. 4, implemented in an example embodiment of the present invention, including the use of a common data model.

FIG. 6 is a schematic illustration of multimodal and connected communication aspects of the system.

FIG. 7 is a schematic illustration of multimodal and connected communication aspects of the system, with a second patient having their medical needs satisfied but using different modalities of communication.

FIG. 8 is a schematic illustration of an example of the resulting ecosystem with the systems of record on the bottom, the common data model, and the app marketplace.

FIG. 9 is a schematic illustration of an example overall system architecture with the systems of record on the bottom of the page and two application stores on the top.

FIG. 10 is a pictorial example of the concept with the two patient centric storefronts populated with direct types of applications.

FIG. 11 is an illustration of customization capabilities of an example system for a member/patient.

### DESCRIPTION OF INVENTION

The invention creates an operational ecosystem that enables the effective integration of health care information into a common repository for the development of applications that will enable progress toward the quadruple aim. The quadruple aim is communally defined as improving the health of populations, enhancing the experience of care for individuals, reducing the per capita cost of health care, and attaining joy in work. The invention recognizes and accounts for the complexities of healthcare by creating storefronts that allow applications with common attributes to be aggregated and distributed effectively. The storefronts create a system that accounts for the unique complexities associated with healthcare such as data privacy management, monitoring responsibilities, availability, and payment nuances. Monitoring responsibilities as used herein refers to the ability and obligation to monitor information submitted by a user or produced by an app (e.g., from a sensor monitored by an app). Secondary monitoring responsibilities as used herein refers to monitoring responsibilities assumed by or imposed on an entity other than the patient.

### Platform Information

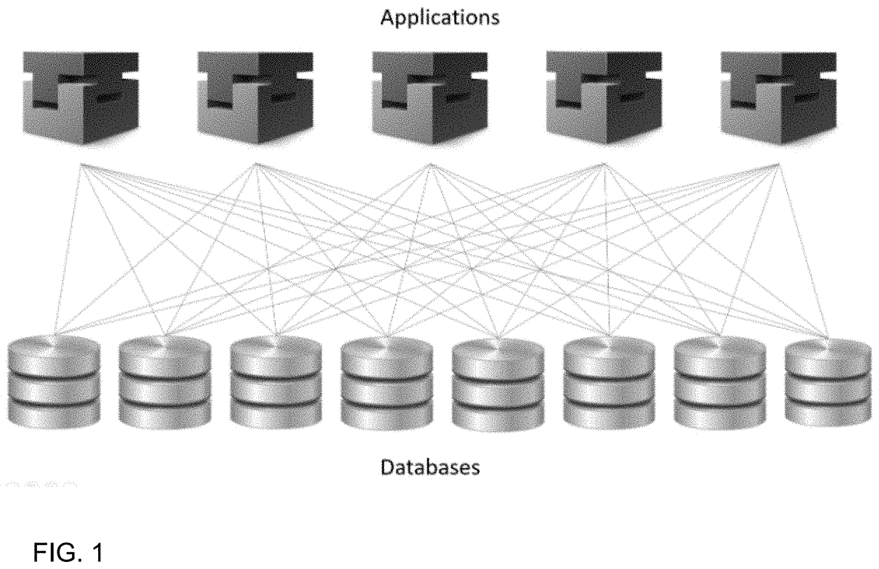

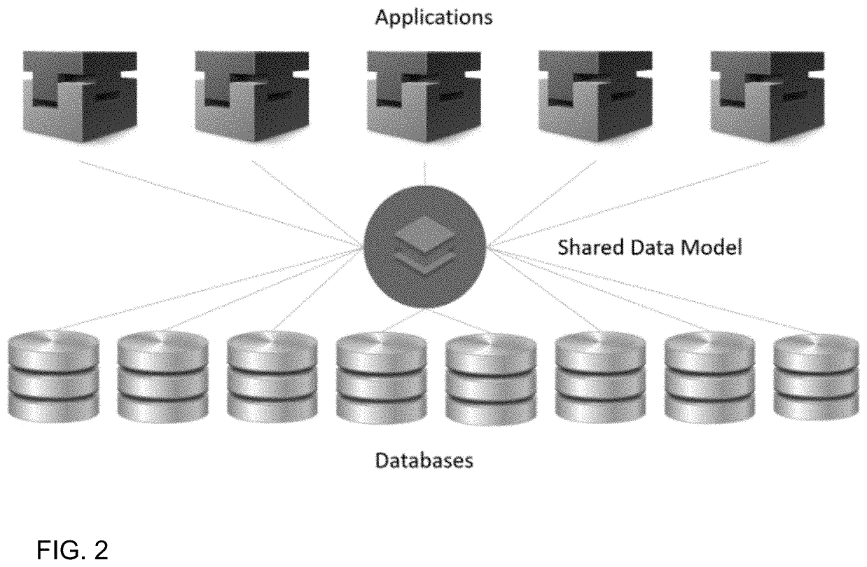

The platform, also referred to as the operating system or ecosystem, is based on a common data model that enables the effective aggregation of information for application development. FIG. 1 is an illustration of applications developed without a common data model. Each application must be connected to each data base. Healthcare currently operates without a common data model. This creates issues often referred to as a lack of interoperability in healthcare. FIG. 2 is an illustration of applications built using a shared or common data model. As the number of databases increases, data integrators can bring data from other repositories into the common data model, instead of building a different model for each app. For the purposes of this invention a common data model is used in the broadest manner and results from aggregating or connecting information for a variety of databases, repositories, files or any digital storage capability. A common data model simplifies data management and app development by unifying data into a known form and applying structural and semantic consistency across multiple apps and deployments. The data in the model can be used by many apps and streamlines the creation of other apps that use the data.

Healthcare currently does not have a common data model and operates with silos of information in disparate systems, including the electronic health record (EHR), provider directories, formularies, health plan information, financial information, and repositories that contain information on the social determinants of health. These data repositories are not integrated to create a common data model upon which applications can be developed. Embodiments of the present invention enable the ingestion, abstraction and normalization of health data into usable data repository.

Embodiments of the present invention provide an improved patient (or health plan member) experience by integrating important elements of obtaining healthcare into one connected system that facilitates the completion of health-related activities. Components of the system can include, but are not limited to, health plan information, provider/delivery system information, and medication order fulfillment information. For example, provider/delivery system information can include the electronic medical record, laboratory values, provider notes, allergies, and other medical information specific for the patient. Plan information can include information specific to payment, deductibles, coverage parameters, etc. The need fulfillment block can include information regarding fulfillment of prescriptions, durable medical equipment, externally purchased services such as physical therapy, imaging, etc. The system can enable communication between these important elements in a real-time or near real-time manner. This common data model is then leveraged to create a system of engagement. Healthcare is dominated by systems of record. A “System of Record” is a data repository of an organization that tracks records, categorizes them, applies a retention period, and ensures records are not improperly deleted. An objective of a system of record is to document activities and ensure compliance rules and regulations are being met and followed. Systems of record generally represent business to business interactions. The electronic medical record and claims system are systems of record. In contrast, a system of engagement is customer focused with an objective of helping customers complete their tasks in the most efficient and effective manner possible.

A platform integrating these systems of record to create a common data model can be used to create a system of engagement that leverages customization at the customer, user, and system level. The platform can be considered an operating system that integrates systems of record, largely but not exclusively from healthcare, creates a standardized system of communication with the patient and allows multi-modal communication, and enables bi-directional communication between the systems of record and selected applications.

### Storefronts

An effective healthcare application marketplace has several nuances not present in historical app stores. Some important nuances are associated with purchaser, user, data privacy, distribution, and monitoring responsibility. In the Apple app store, as an example, the purchaser and user of the app are the same person, and health data provided by the patient or created by the patient is used by the patient and controlled by the patient. The responsibility to monitor or response to the data is also held by the patient. For example, the Wahoo fitness application is an exercise monitoring application purchased, downloaded, and monitored by the individual. An application marketplace for health care necessitates refinement and awareness that other parties and complexities exist in purchasing, use, data privacy and monitoring.

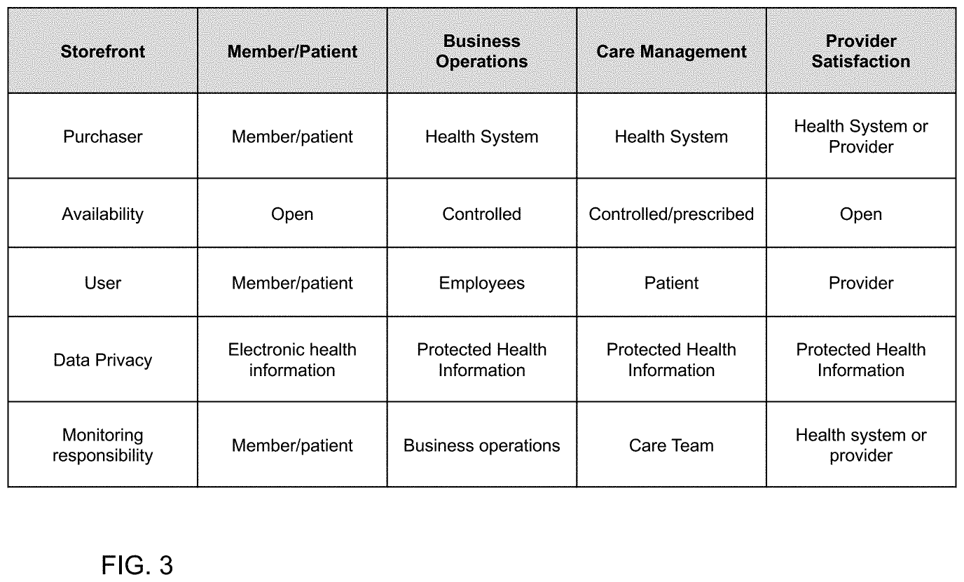

The invention creates a structure for defining and creating different storefronts. A storefront is defined as a collection of applications that have common or related attributes. For the purposes of illustration, four storefronts will be defined, and five attributes will be used for the associated storefronts. The invention includes systems with more or fewer storefronts, and using more or fewer attributes. The example storefronts are member/patient, business operations, care management, and provider satisfaction (also referred to as provider efficiency) applications. The attributes used to define these storefronts are purchaser, availability, user, data privacy and monitoring responsibility. FIG. 3 is a table illustrating the distinct attribute differences between the storefronts. The purchaser of the application is the entity that supports the use of the application via purchase. The availability is associated with the mechanism of distribution, e.g., is the app available on the open market or is it controlled by the healthcare system. The user of the application is the entity that uses the application to create information of value. Data privacy is the mechanism for ensuring that health information is managed in a trusted and appropriate manner. Monitoring responsibility indicates the party that has an obligation to examine, interpret and react to the data generated.

The monitoring responsibility creates an important differentiation as it impacts availability, distribution and privacy. If the application generates or obtains data that places an obligation on the healthcare system to respond, then the use of these applications is controlled by the responsible healthcare system. If data generated or obtained for the application is for the use of the customer and the responsibility to monitor and react to the information resides with the customer, then these applications can exist in a public application store. If data generated or obtained for the application is for improving a business process, then the responsibility to monitor and react to the information resides with the business owner. The ability to designate and manage applications with different responsibility obligations as well as purchasing mechanisms within an integrated ecosystem is a unique attribute of the present invention.

Applications that have an associated monitoring responsibility are not made available via an open app marketplace but are controlled by the party responsible for monitoring and reacting to the data, typically a health system or provider group. A health system is an entity or organization that provides health care or related services to patients, as an example a hospital, integrated delivery system, or a system integrating financing and delivery. A provider group is an entity or organization or individual that provides health care or related services to patients, as an example a group of surgeons who have affiliated their practice. As an example, the responsible party can control access to such applications via prescription to the digital tools housed in a health system app store. The health system app store is controlled by the healthcare system and is populated with applications that facilitate care delivery and wellness and place a monitoring or response obligation on the system. Because the applications in the health system store create a response and monitoring obligation by the health system, the health system controls which of such applications are made available to the patient/customer. Example applications include chronic care monitoring applications. If the patient is experiencing an exacerbation of a chronic condition, an appropriate application can generate information reflecting a problem. The healthcare system via monitoring of the information can proactively engage the patient for better outcomes and improved health. Another example is related to fulfilment of medically necessary equipment or services. For example, an application can be used to monitor oxygen consumption. At the point the patient is about to run out (or reaches some other threshold), the system manages the fulfillment of the needed oxygen, e.g., by initiating a delivery of oxygen to the patient's residence. Additionally, the system can monitor consumption rate and if excessive (or below expectations) it might indicate a pending medical problem or a compliance problem. Other examples include diabetes management, COPD management, immunization management, advanced care planning, home health coordination, and virtual care.

The ecosystem is constructed to allow the effective management of health data privacy. As noted in FIG. 3 the various storefronts manage privacy differently based on government regulations. Electronic health information (EHI) is controlled the patient/consumer, specifically the patient/consumer approves the release of EHI either for their own use or for the use of a third party. Protected Health Information (PHI) is shared for the purpose of the payment, treatment and operations between covered entities (business associates) under HIPAA. Protected health information (PHI), also referred to as personal health information, generally refers to demographic information, medical histories, test and laboratory results, mental health conditions, insurance information, and other data that a healthcare professional collects to identify an individual and determine appropriate care. The platform will have access to PHI, so criteria to ensure that it is effectively protected must be satisfied by any applications interacting with the common data model. Data privacy is an important attribute that will distinguish the operational characteristics of a given storefront due to difference in EHI and PHI. In an example embodiment, the ecosystem utilizes a data sharing datagram. The data sharing datagram is a tool that defines what data elements are needed for the application and will restrict access to those data elements not needed. Such a data control method allows access to appropriate data but does not provide open access to all data. In an example embodiment, each storefront has a defined data sharing datagram with customization as needed.

The invention manages attribute differences between different applications in a coordinated and coherent manner such that the applications can have maximal availability and impact, while managing privacy concerns.

Embodiments of the present invention provide an improved patient/member experience by dynamically matching the modality of communication with the complexity of the user needs. Based upon user needs, the dynamic allocation system can recommend the best communication modality for resolving the user's needs and/or questions. The initial selection of communication modality can change over the interaction. As the character of the interaction changes, the system can dynamically suggest another modality for communication.

### Example Embodiment

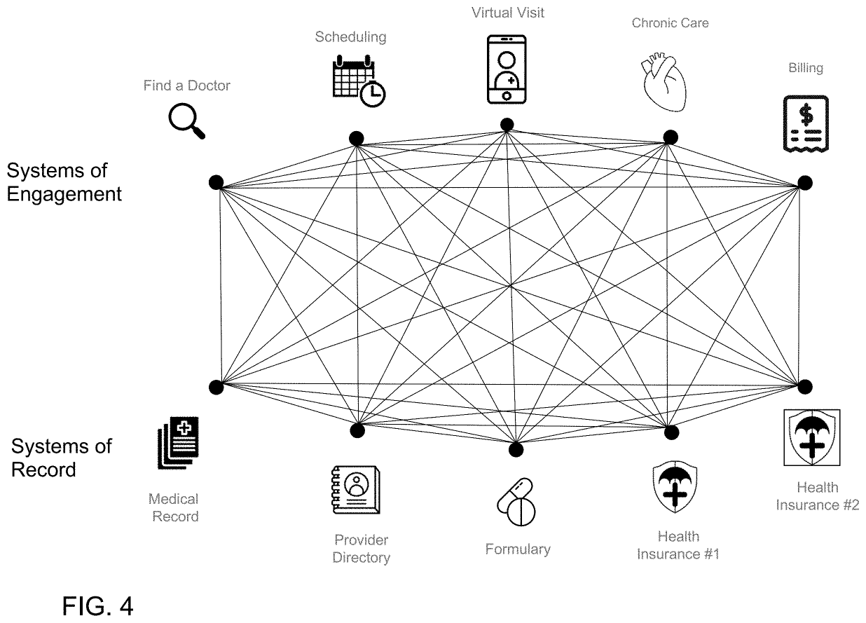

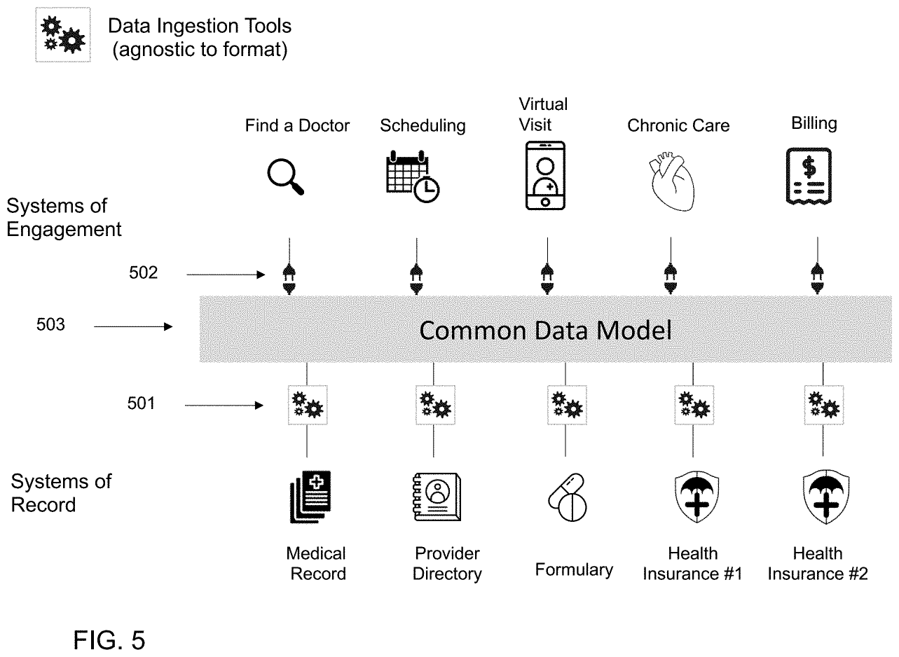

FIG. 4 shows the current interconnections of an example system without benefit of the present invention and how the associated parts communicate. FIG. 5 shows similar functionality implemented in an example embodiment of the present invention, including the use of a common data model. Ingestion tools (501) that enable the ingestion, transfer, abstraction and normalization of health data from the systems of record to the common data model are pictorially represented. For example, there exist multiple types of electronic medical records so the ingestion tools will appropriately map the information onto the common data model. The ability to plug applications into the common data model is pictorially represented (502). The capability to integrate applications into the ecosystem without prolonged integration times is a significant benefit of the system. This capacity reduces integration time and will create additional options for customization. The common data model (503) is populated with the information needs by the applications. The common data model includes but is not limited to health plan information, provider/delivery system information, social determinants of health, financial information, and medication order fulfillment information. For example, provider/delivery system information contained in the EHR can include the electronic medical record, laboratory values, provider notes, allergies, and other medical information specific for the patient. Plan information can include information specific to payment, deductibles, coverage parameters, etc. The need fulfillment block can include information regarding fulfillment of prescriptions, durable medical equipment, externally purchased services such as physical therapy, imaging, etc. An element of the example embodiment is the intercommunication between these important elements as well as others that can define the healthcare ecosystem for a given patient/member. The ability to move information between these disparate systems is an important benefit of the system.

The communication architecture allows the transfer of information between key systems such as the electronic medical record. These existing systems can communicate through an application programming interface (APIs). An API is a set of subroutine definitions, protocols, and tools for building application software. In general terms, it is a set of defined methods of communication between various software components on onto a system bus. The combination of established communication bus and robust APIs provide the building blocks for a communication platform with a “plug and play” capability. An enterprise service bus is a communication system that has applicability to such an integrated system. An enterprise service bus (ESB) applies the design concept of modern operating systems to independent services running on or within networks of disparate and independent computers. Like concurrent operating systems, an ESB provides commodity services in addition to adoption, translation and routing of client requests to appropriate answering services. Functions of an enterprise service bus include routing messages between services, monitoring and control routing of message exchange between services, resolving contention between communicating service components, and providing commodity services like event handling, data transformation and mapping, message and event queuing and sequencing, security or exception handling, protocol conversion and enforcing proper quality of communication service. Many options exist for moving information between various system of record.

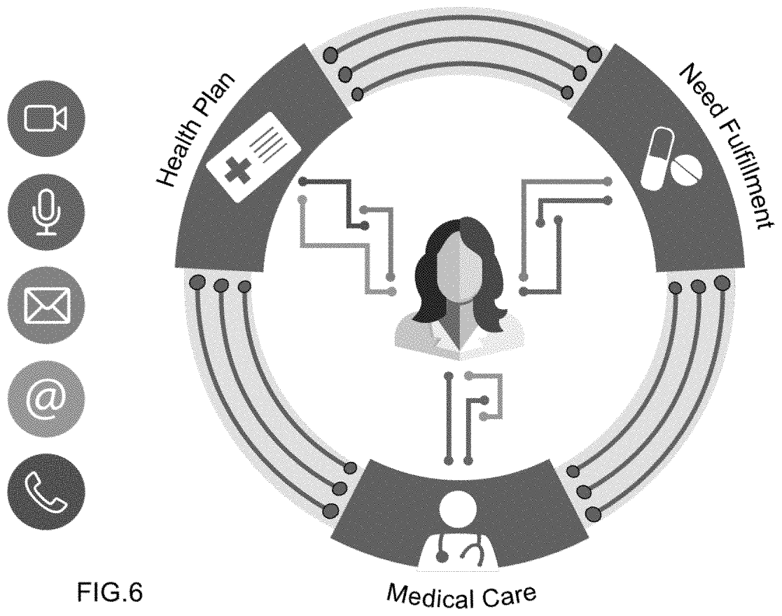

FIG. 6 shows multimodal and connected communication aspects of the system. As illustrated, the patient might seek to interact with her health plan via text but might initiate a second discussion via email with the transition to phone. The system can be agnostic to the location of information and simply provide a seamless communication system satisfying the patient's needs. Of note, the system connects to the appropriate information via the common data model and coordinates the communication between disparate databases/programs/systems in a way that creates an extraordinary user experience. The resulting system is dynamic, flexible and responsive to the patient's needs.

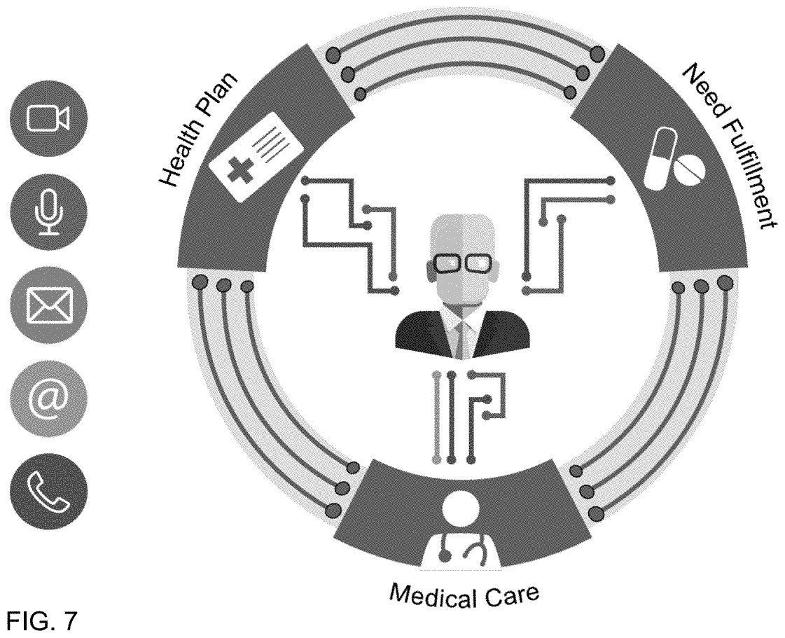

FIG. 7 shows a second patient having their medical needs satisfied but using different modalities of communication. As depicted in the figure, the representative individual is older and prefers phone communication. The resulting integrated communication system allows the patient to utilize their modality of preference while creating a continuous transfer of information between associated systems. For example, the patient may receive a new medication from the healthcare provider. Within the same system and even at the same point in time, the patient can determine the expense of the medication and then have the medication refill without difficulty.

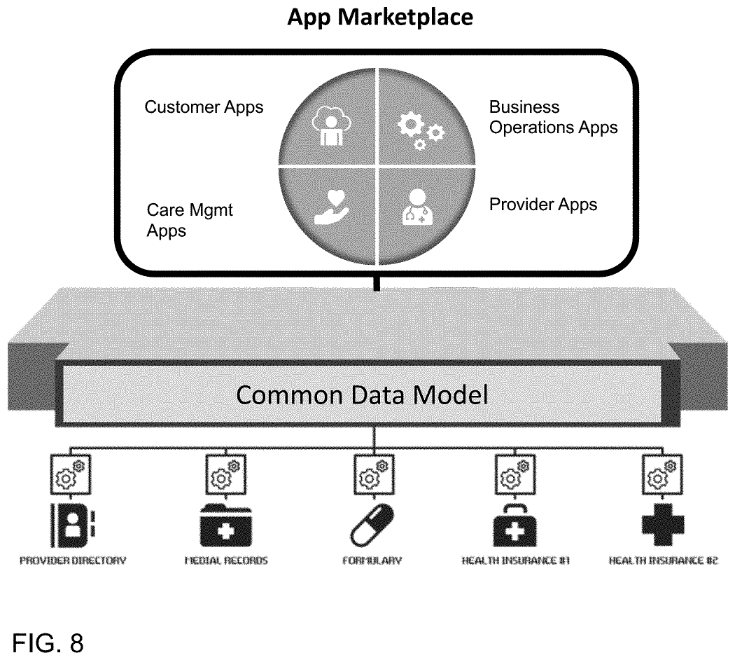

FIG. 8 is an example illustration of the resulting ecosystem with the systems of record on the bottom, the common data model, and the app marketplace. The app marketplace shows the 4 example storefronts defined and representing different attributes. It is recognized that the marketplace can be expanded with different storefronts and some implementations might use a single storefront where the applications are not bundled by attributes but are self-defined units. The resulting ecosystem enables third-party programmers to create specific tools that interface with the common data model. Allowing new applications allows the system to grow organically. For example, a new application or system associated with a chronic disease condition can leverage the common data model or the established protocols of the enterprise service bus such that an integrated communication and data transfer system is available to the patient and provider. Example add-on tools include medication management, remote monitoring, video visits, virtual exams, smart speakers, messaging platforms, reminder platforms, navigation systems, emergency response systems, payment systems, registration systems, patient intake forms, etc. The ability to augment the overall capability of the system through application development is a useful capacity of the proposed invention. Such a capacity allows the system to be expanded using third-party applications with overall governance of communication protocol being managed by the service bus, or common data model.

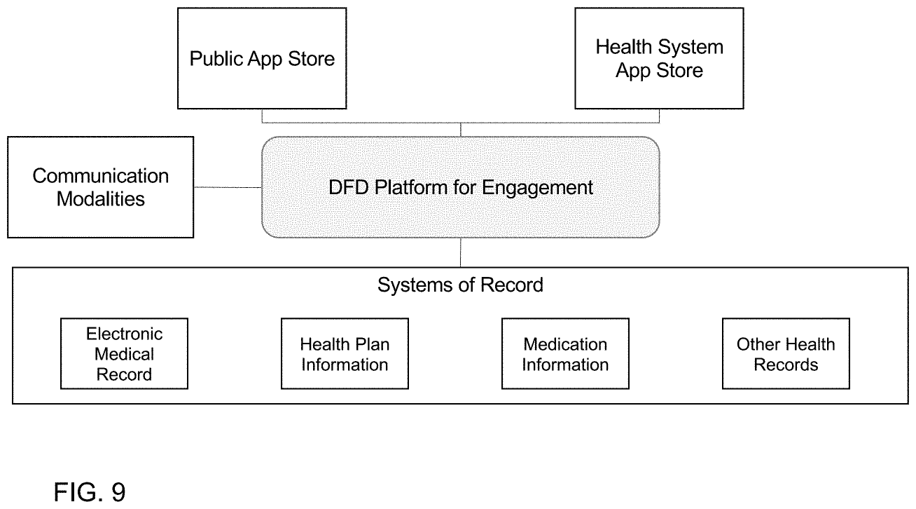

FIG. 9 show an example overall system architecture with the systems of record on the bottom of the page and two application stores on the top. The system leverages principles of modularity. Modularity refers to the concept of making multiple modules and then linking and combining them to form a complete system. Modularity enables re-usability and minimizes duplication. The customer experience of a modular system can be improved by using a common communication or help system. A patient might be experiencing a problem with one application, but the source of the problem is another application in the ecosystem. Thus, a common communication and help mechanism can help with overall product performance and enhancement.

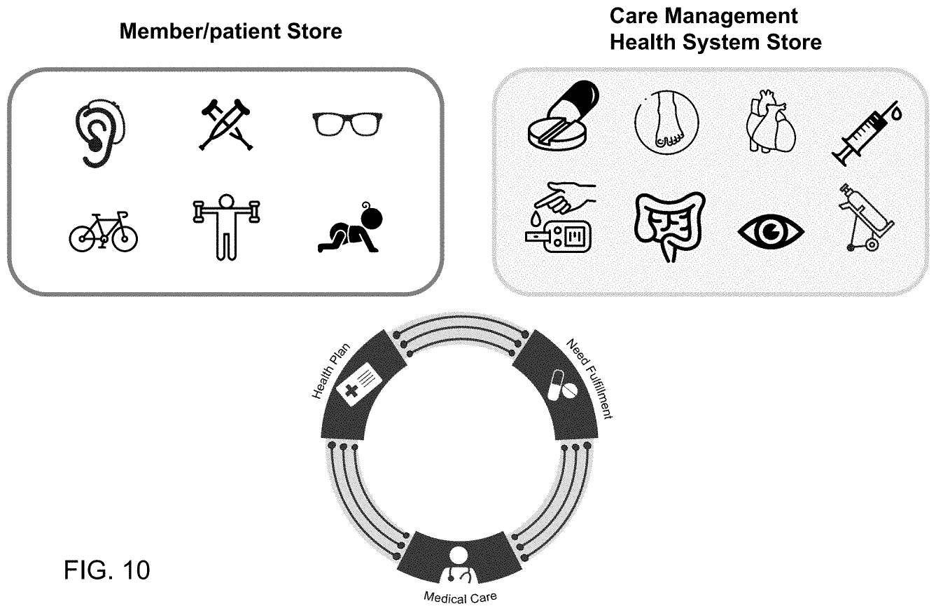

FIG. 10 shows a pictorial example of the concept with the two patient centric storefronts populated with direct types of applications. As noted, the member/patient storefront contains applications that generate information for use by the customer. As used herein, a storefront “contains” an app when the app is accessible using the storefront, as examples by downloading the app from the storefront to a personal computer, handheld device such as a smartphone or smartwatch, or a medical instrument; or by accessing functionality of the app via a network communication such as using a browser communicating with a remote computing system running the app. The care management storefront contains applications that create data that is used by both the customer (patient/member) and the health system, with a corresponding monitoring and response obligation that resides with the healthcare system

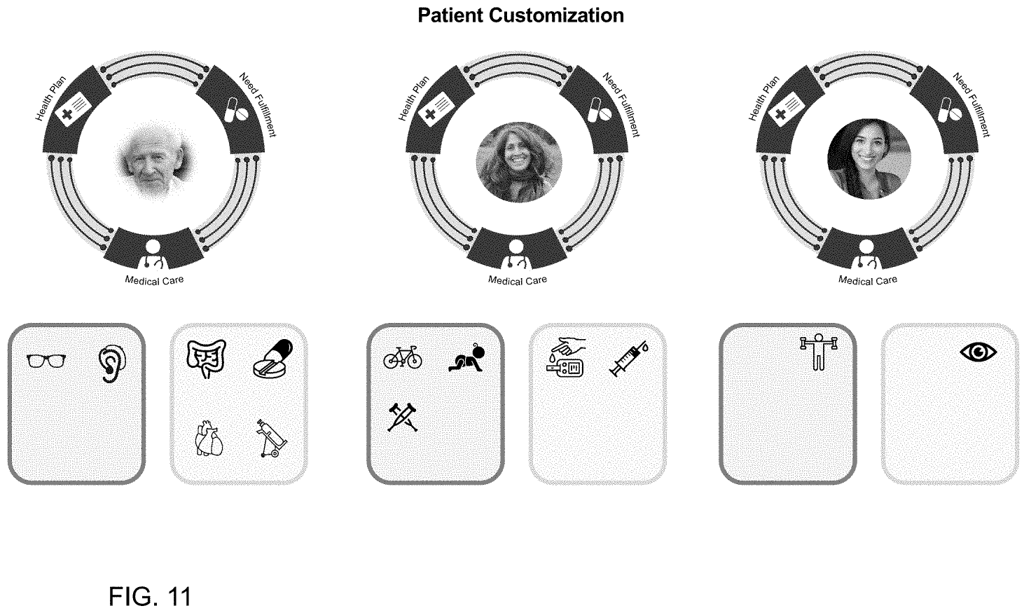

FIG. 11 shows customization capabilities of the system for the member/patient. The picture of the older gentleman on the left depicts a patient that has several chronic conditions and is being proactively monitored by the healthcare system. Additionally, he has applications from the member/patient store associated with vision and his hearing aid.

The “mom” in the middle has populated her system with applications pertinent to her medical needs. She has applications associated with monitoring her fitness and the development of her young son. She also has application provide by the health system to help with her daughter that has diabetes. These applications help with glucose control and the automatic ordering of insulin.

The last patient, the “young millennial” on the right has applications specific for her needs. She is a has a general fitness application to earn reward points with her health plan and a medical application to manage a pre-existing eye condition.

The result is an ecosystem that creates a differentiated customer experience though seamless integration of health record information that is then utilized by applications to help the customer get their medical needs satisfied. The ability to customize in a controlled but individualized manner creates a system that is differentiated from prior approaches.

Implementation. Methods and apparatuses according to the present invention can be implemented using multiple computers connected in a distributed network. Traditionally, a computer program consists of a finite sequence of computational instructions or program instructions. It will be appreciated that a programmable apparatus (i.e., computing device) can receive such a computer program and, by processing the computational instructions thereof, produce a further technical effect.

A programmable apparatus includes one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computer can include any and all suitable combinations of a special-purpose computer, programmable data processing apparatus, processor, processor architecture, and so on.

It will be understood that a computer can include a computer-readable storage medium and that this medium can be internal or external, removable and replaceable, or fixed. It will also be understood that a computer can include a Basic Input/Output System (BIOS), firmware, an operating system, a database, or the like that can include, interface with, or support the software and hardware described herein.

Embodiments of the systems as described herein are not limited to applications involving conventional computer programs or programmable apparatuses that run them. It is contemplated, for example, that embodiments of the invention as claimed herein could include an optical computer, quantum computer, analog computer, or the like.

Regardless of the type of computer program or computer involved, a computer program can be loaded onto a computer to produce a particular machine that can perform any and all of the depicted functions. This particular machine provides a means for carrying out any and all of the depicted functions.

Any combination of one or more computer readable medium(s) can be utilized. The computer readable medium can be a computer readable signal medium or a computer readable storage medium. A computer readable storage medium can be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium can be any tangible medium that can contain, or store a program for use by or in connection with an instruction execution system, apparatus, or device.

According to an embodiment of the present invention, a data store can be comprised of one or more of a database, file storage system, relational data storage system or any other data system or structure configured to store data, or example in a relational manner. In an embodiment of the present invention, the data store can be a relational database, working in conjunction with a relational database management system (RDBMS) for receiving, processing and storing data. In an embodiment, the data store can comprise one or more databases for storing information related to the processing of information as well one or more databases configured for storage and retrieval of information.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium can include a propagated data signal with computer readable program code embodied therein, for example, in baseband or as part of a carrier wave. Such a propagated signal can take any of a variety of forms, including, but not limited to, electro-magnetic, optical, or any suitable combination thereof. A computer readable signal medium can be any computer readable medium that is not a computer readable storage medium and that can communicate, propagate, or transport a program for use by or in connection with an instruction execution system, apparatus, or device.

Program code embodied on a computer readable medium can be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof can be implemented as parts of a monolithic software structure, as standalone software modules, or as modules that employ external routines, code, services, and so forth, or any combination of these. All such implementations are within the scope of the present disclosure.

In view of the foregoing, it will now be appreciated that elements of the block diagrams and flowchart illustrations support combinations of means for performing the specified functions, combinations of steps for performing the specified functions, program instruction means for performing the specified functions, and so on.

It will be appreciated that computer program instructions can include computer executable code. A variety of languages for expressing computer program instructions are possible, including without limitation C, C++, Java, JavaScript, assembly language, Lisp, HTML, Perl, and so on. Such languages can include assembly languages, hardware description languages, database programming languages, functional programming languages, imperative programming languages, and so on. In some embodiments, computer program instructions can be stored, compiled, or interpreted to run on a computer, a programmable data processing apparatus, a heterogeneous combination of processors or processor architectures, and so on. Without limitation, embodiments of the system as described herein can take the form of web-based computer software, which includes client/server software, software-as-a-service, peer-to-peer software, or the like.

In some embodiments, a computer enables execution of computer program instructions including multiple programs or threads. The multiple programs or threads can be processed more or less simultaneously to enhance utilization of the processor and to facilitate substantially simultaneous functions. By way of implementation, any and all methods, program codes, program instructions, and the like described herein can be implemented in one or more thread. The thread can spawn other threads, which can themselves have assigned priorities associated with them. In some embodiments, a computer can process these threads based on priority or any other order based on instructions provided in the program code.

Unless explicitly stated or otherwise clear from the context, the verbs “execute” and “process” are used interchangeably to indicate execute, process, interpret, compile, assemble, link, load, any and all combinations of the foregoing, or the like. Therefore, embodiments that execute or process computer program instructions, computer-executable code, or the like can suitably act upon the instructions or code in any and all of the ways just described.

The functions and operations presented herein are not inherently related to any particular computer or other apparatus. It is possible to modify or customize general-purpose systems to be used with programs in accordance with the teachings herein, or it might prove convenient to construct more specialized apparatus to perform the required method steps. The required structure for a variety of these systems will be apparent to those of skill in the art, along with equivalent variations. In addition, embodiments of the invention are not described with reference to any particular programming language. It is appreciated that a variety of programming languages can be used to implement the present teachings as described herein, and any references to specific languages are provided for disclosure of enablement and best mode of embodiments of the invention. Embodiments of the invention are well suited to a wide variety of computer network systems over numerous topologies. Within this field, the configuration and management of large networks include storage devices and computers that are communicatively coupled to dissimilar computers and storage devices over a network, such as the Internet.

Throughout this disclosure and elsewhere, block diagrams and flowchart illustrations depict methods, apparatuses (i.e., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general purpose hardware specialized through computer instructions; and so on—any and all of which can be generally referred to herein as a “circuit,” “module,” or “system.”

While the foregoing drawings and description set forth functional aspects of the disclosed systems, no particular arrangement of software for implementing these functional aspects should be inferred from these descriptions unless explicitly stated or otherwise clear from the context.

The functions, systems and methods herein described can be utilized and presented in a multitude of languages. Individual systems can be presented in one or more languages and the language can be changed with ease at any point in the process or methods described above. One of ordinary skill in the art would appreciate that there are numerous languages the system could be provided in, and embodiments of the present invention are contemplated for use with any language.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

We claim:

1. A system for providing a plurality of health-related apps to a plurality of different types of users, comprising:
(a) a data access system, storing at least two of the following: directory of providers, patient medical records, formulary, health insurance plan coverage records; wherein each of the foregoing is accessible using a common data model;
(b) a plurality of storefronts, comprising at least two of the following: (1) a customer app storefront, accessible to individual health plan members, patients, or both, and containing apps that accept health-related data from the user and do not access PHI associated with any other individual; (2) a business operations storefront, accessible to individuals authorized by business operations of one or more health system, and containing apps that access PHI of members of the health system; (3) a care management storefront, accessible to individuals authorized by a health care provider or health insurance plan, and containing apps that collect or allow input of PHI of the individual and communicate said PHI to a health care provider or health insurance plan; and (4) a provider storefront, accessible to one or more health care providers, and containing apps that access of PHI of an individual from a health system, and communicate said PHI to one or more health care providers or health insurance plans.

2. The system of claim 1, wherein the plurality of storefronts comprises at least three of the storefronts listed.

3. The system of claim 1, wherein the plurality of storefronts comprises all four of the storefronts listed.

4. The system of claim 1, wherein the customer app storefront contains apps that are downloadable to a personal smart device.

5. The system of claim 4, wherein the customer app storefront contains apps that are downloadable to at least one of a smart phone, a smart watch, and a tablet.

6. The system of claim 1, wherein the date storage system comprises a plurality of distinct data storage facilities, each controlled by a distinct health care system and containing PHI corresponding to members of the health care system.

7. The system of claim 6, wherein each data storage facility has a distinct data storage model, and further comprising software that translates data storage actions and data retrieval actions between the common data model and the data storage model of the distinct storage facility to which the data storage or data retrieval action pertains.

8. The system of claim 1, wherein an individual can access an app from the care management storefront upon receiving a prescription for the app from a health care provider.

9. A system for providing a plurality of health-related apps to a plurality of different types of users, comprising:
(a) a common data model with access to at least two of the following: a directory of providers, patient medical records, a formulary, health insurance plan coverage records;
(b) a plurality of storefronts that provide access to apps, wherein the apps are available in storefronts based on how the app implements privacy management of the patient's data and how the app implements secondary monitoring obligations relative to information from the app.

10. The system of claim 9, wherein the plurality of storefronts comprises a first storefront wherein apps provide for privacy managed by a health system, and a second storefront wherein apps provide for privacy managed by the patient.

11. The system of claim 9, wherein the plurality of storefronts comprises a first storefront wherein apps provide for secondary monitoring by a health system, and a second storefront wherein apps provide for data monitoring by a patient.

12. The system of claim 9, wherein at least one storefront contains apps that are downloadable to a personal smart device, including a smart phone, smart watch, and/or a tablet.

13. The system of claim 9, wherein the plurality of storefronts comprises at least two of the following: (1) a customer app storefront, accessible to individual health plan members, patients, or both, and containing apps that accept health-related data from the user and do not access PHI associated with any other individual; (2) a business operations storefront, accessible to individuals authorized by business operations of one or more health systems, and containing apps that access PHI of members of the health system; (3) a care management storefront, accessible to individuals authorized by a health care provider or health insurance plan, and containing apps that collect or allow input of PHI of the individual and communicate said PHI to a health care provider or health insurance plan; and (4) a provider storefront, accessible to one or more health care providers, and containing apps that access PHI of an individual patient from a health system, and communicate said PHI to one or more health care providers or health insurance plans.

14. The system of claim 13, wherein the customer app storefront contains apps that are downloadable to at least one of a smart phone, a smart watch, and a tablet.

15. The system of claim 9, wherein the common data model is configured to access a plurality of distinct data storage facilities, each controlled by a distinct health care system and containing PHI corresponding to members of the health care system.

16. The system of claim 15, wherein the common data model uses software that translates stored health data storage actions and data retrieval actions between the common data model and the data storage model of the distinct storage facility to which the data storage or data retrieval action pertains.

17. The system of claim 9, wherein an individual can access an app from the care management storefront upon receiving a prescription for the app from a health care provider.
